(19) United States
(12) Patent Application Publication
(10) Pub. No.: US 2026/0026955 A1
(43) Pub. Date: Jan. 29, 2026

(54) METHODS OF DESIGNING AND MANUFACTURING CUSTOM-FIT WEARABLE DEVICES

(71) Applicant: XO Armor Technologies, Inc., Auburn, AL (US)

(72) Inventors: Jacob Larson, Opelika, AL (US); Scot Carpenter, Opelika, AL (US); John Alan Frost, Birmingham, AL (US); Joseph Bingold, Portland, OR (US); Michael Zabala, Waverly, AL (US)

(21) Appl. No.: 19/278,408

(22) Filed: Jul. 23, 2025

Related U.S. Application Data
(60) Provisional application No. 63/675,441, filed on Jul. 25, 2024.

(51) Int. Cl. A61F 5/01; A61B 5/107; B33Y 50/02; B33Y 80/00; G05B 19/4099; G06T 19/20

(52) U.S. Cl. CPC A61F 5/0111; B33Y 50/02; B33Y 80/00; G05B 19/4099; G06T 19/20; A61B 5/1077; G05B 2219/35134; G05B 2219/49023; G06T 2219/2004; G06T 2219/2016

(57) ABSTRACT

Methods and systems are disclosed for manufacturing a custom-fit wearable device. In general, the methods and systems include generating a three-dimensional model of a body portion, identifying one or more anatomical landmarks, and mapping a curve onto the model based at least in part on those landmarks to define a continuous surface. Thickness and an offset may be applied to the surface. The resulting model may then be used to control an additive manufacturing process to produce the custom-fit wearable device.

### CROSS-REFERENCES

The following applications and materials are incorporated herein by reference, in their entireties, for all purposes: U.S. Provisional Patent Application No. 63/675,441, filed Jul. 25, 2024.

### FIELD

This disclosure relates to systems and methods for manufacturing custom-fit wearable devices.

### SUMMARY

The present disclosure provides systems, apparatuses, and methods relating to designing and manufacturing custom-fit wearable devices.

A method of manufacturing a custom-fit wearable device in accordance with aspects of the present disclosure may include identifying one or more anatomical landmarks on the three-dimensional model; mapping a curve onto the three-dimensional model based at least in part on the one or more anatomical landmarks, such that a surface of a wearable device model is defined by a continuous area of the three-dimensional model bound by the curve; adding thickness to the surface of the wearable device model; and controlling an additive manufacturing process to manufacture the custom-fit wearable device based on the wearable device model.

A data processing system for generating a wearable device model in accordance with aspects of the present disclosure may include one or more processors; a memory; and a plurality of instructions stored in the memory and executable by the one or more processors to: receive as input a scan of an anatomical region to generate a three-dimensional model of a body portion; identify one or more anatomical landmarks on the three-dimensional model; map a curve onto the three-dimensional model based at least in part on the one or more anatomical landmarks, such that a surface of a wearable device model is defined by a continuous area of the three-dimensional model bound by the curve; add a thickness to the surface of the wearable device model; and control an additive manufacturing process to manufacture a custom-fit wearable device based on the wearable device model.

A computer implemented method of generating a virtual model of a wearable device in accordance with aspects of the present disclosure may include receiving a scan of an anatomical region; converting the scan to a continuous surface model; aligning and scaling a three-dimensional curve corresponding to a wearable device to the continuous surface based on one or more landmarks of the continuous surface model; defining edge boundaries of the wearable device based on the three-dimensional curve; trimming the continuous surface model to the edge boundaries to define a trimmed surface; and applying thickness and offset to the trimmed surface to generate a virtual model of the wearable device.

Features, functions, and advantages may be achieved independently in various embodiments of the present disclosure, or may be combined in yet other embodiments, further details of which can be seen with reference to the following description and drawings.

### Introduction

Wearable devices, such as braces, orthoses, orthopedic supports, and protective equipment are often utilized for alleviating pain, improving mobility, limiting unwanted motion, and providing protection. Despite these benefits, wearable devices often suffer from deficiencies in fit, which may limit their effectiveness. Traditional custom fitting methods, such as plaster casting, often are time consuming, cost prohibitive, and unable to accommodate natural biomechanical motion of joints.

### BRIEF DESCRIPTION OF THE DRAWINGS

FIG. 1 is a flow chart depicting steps of an illustrative method for designing and manufacturing a wearable device in accordance with aspects of the present disclosure.

FIG. 2 depicts an example three-dimensional model of a lower leg suitable for use with the method of FIG. 1.

FIG. 3 depicts an example three-dimensional curve suitable for use with the method of FIG. 1 in a first orientation.

FIG. 4 depicts the example three-dimensional curve of FIG. 3 in a second orientation.

FIG. 5 depicts the example three-dimensional curve of FIG. 3 in a third orientation.

FIG. 6 depicts the example three-dimensional curve of FIG. 3 in a fourth orientation.

FIG. 7 depicts the example three-dimensional curve of FIG. 3 in a fifth orientation.

FIG. 8 depicts the example three-dimensional curve of FIG. 3 mapped onto the three-dimensional scan of FIG. 2.

FIG. 9 depicts the three-dimensional scan of FIG. 2 with a wearable device surface defined by the mapped curve of FIG. 8.

FIG. 10 depicts the wearable device surface of FIG. 9, with excess portions of the scan removed.

FIG. 11 depicts joint rotation axes of an ankle of the three-dimensional scan of FIG. 2.

FIG. 12 depicts an example of a virtual rotation of a portion of the three-dimensional scan of FIG. 2 about a joint axis.

### DETAILED DESCRIPTION

Various aspects and examples of custom wearable devices, including related methods of manufacture and design, are described below and illustrated in the associated drawings. Unless otherwise specified, a wearable device in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

### Definitions

The following definitions apply herein, unless otherwise indicated.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“AKA” means “also known as,” and may be used to indicate an alternative or corresponding term for a given element or elements.

“Flexible” describes a material or structure configured to respond to normal operating loads (e.g., when compressed, stretched, expanded, bent, etc.) by deforming elastically and returning to an original shape or position when unloaded.

“Rigid” describes a material or structure configured to be stiff, non-deformable, or substantially lacking in flexibility under normal operating conditions.

“Model” refers to a digital representation of an object's shape and structure in three dimensions, e.g., as defined by geometric data such as vertices, edges, and/or surfaces.

“Processing logic” describes any suitable device(s) or hardware configured to process data by performing one or more logical and/or arithmetic operations (e.g., executing coded instructions). For example, processing logic may include one or more processors (e.g., central processing units (CPUs) and/or graphics processing units (GPUs)), microprocessors, clusters of processing cores, FPGAs (field-programmable gate arrays), artificial intelligence (AI) accelerators, digital signal processors (DSPs), and/or any other suitable combination of logic hardware.

A “controller” or “electronic controller” includes processing logic programmed with instructions to carry out a controlling function with respect to a control element. For example, an electronic controller may be configured to receive an input signal, compare the input signal to a selected control value or setpoint value, and determine an output signal to a control element (e.g., a motor or actuator) to provide corrective action based on the comparison. In another example, an electronic controller may be configured to interface between a host device (e.g., a desktop computer, a mainframe, etc.) and a peripheral device (e.g., a memory device, an input/output device, etc.) to control and/or monitor input and output signals to and from the peripheral device.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

### Overview

In general, disclosed methods for designing and manufacturing custom-fit wearable devices, such as casts, braces, splints, pads, etc., utilize three-dimensional scans of the corresponding anatomical region.

In general, the disclosed methods include obtaining a three-dimensional scan of a selected anatomical region, converting the three-dimensional scan into a three-dimensional surface model, mapping a three-dimensional curve corresponding to a desired wearable device onto the surface of the surface model, utilizing the mapped curve to define the edge boundaries of the wearable device, trimming away excess portions of the surface model to define a virtual model of the wearable device, and in some examples, adding an offset to the wearable device model. The virtual model of the wearable device then can be additively manufactured to produce the wearable device.

Furthermore, disclosed methods for designing and manufacturing custom-fit wearable devices may utilize digital repositioning of the surface model prior to manufacture, e.g., to achieve a different (for example, preferred) joint angle. Additionally, or alternatively, disclosed methods may utilize one or more techniques to incorporate a desired amount and degree of flexibility. For example, flexibility may be achieved by adjusting material properties, material thicknesses, wearable model geometries, etc.

### Examples, Components, and Alternatives

The following sections describe selected aspects of illustrative methods of designing and manufacturing wearable devices, as well as related systems and/or methods. The examples in these sections are intended for illustration and should not be interpreted as limiting the scope of the present disclosure. Each section may include one or more distinct embodiments or examples, and/or contextual or related information, function, and/or structure.

### A. Illustrative Method for Designing and Manufacturing Custom-Fit Wearable Devices

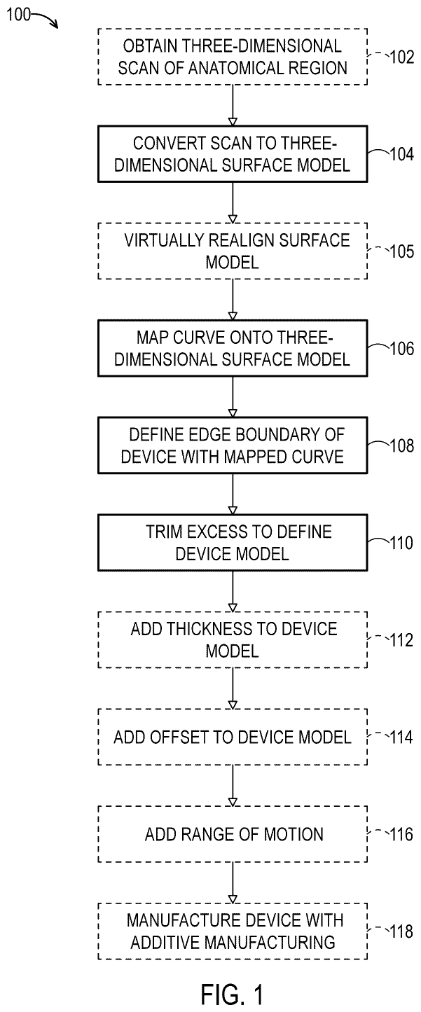

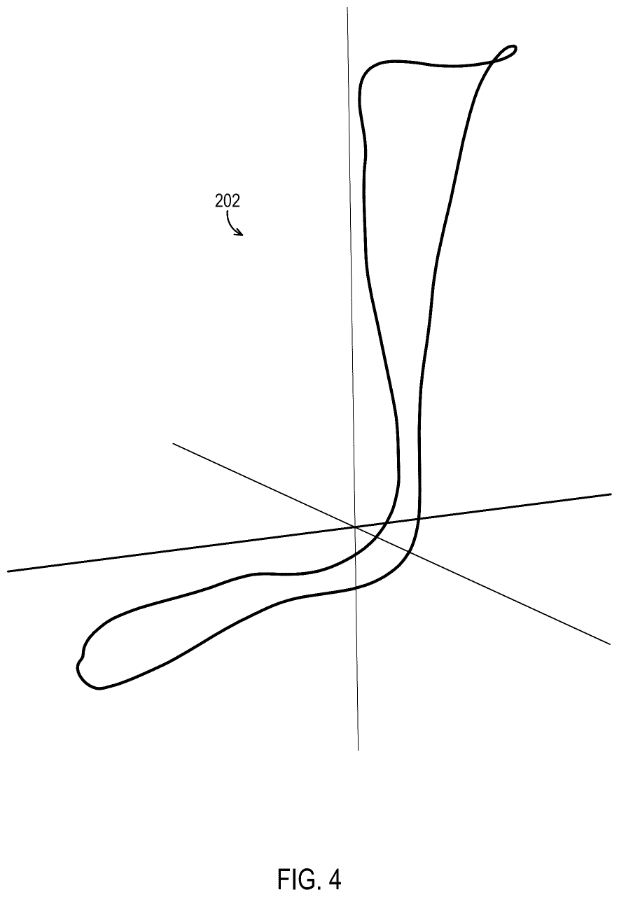

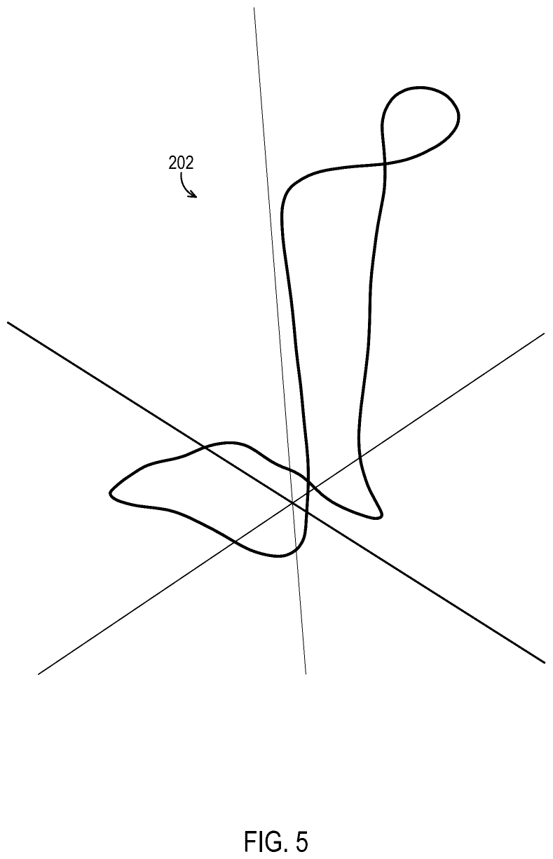

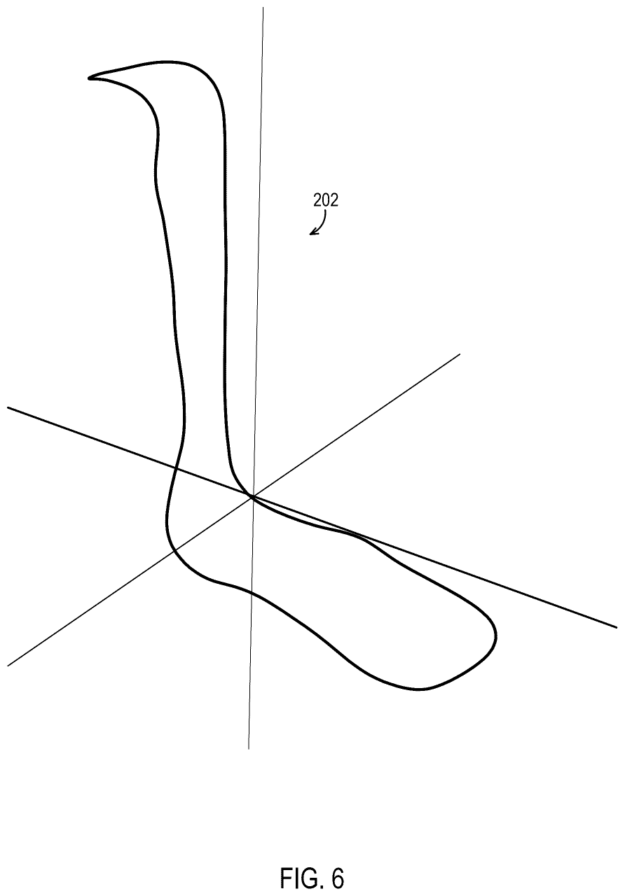

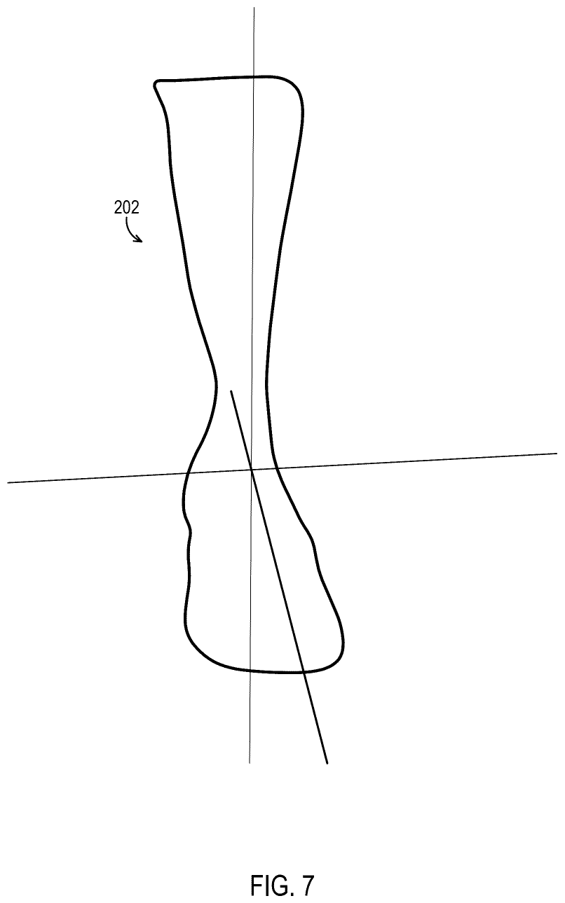

This section describes steps of an illustrative method 100 for designing and manufacturing custom-fit wearable devices, such as casts, braces, splints, pads, etc. See FIGS. 1-12. Where appropriate, reference may be made to components, systems, and/or other steps that may be used in carrying out each step. These references are for illustration, and are not intended to limit the possible ways of carrying out any particular step of the method.

FIG. 1 is a flowchart illustrating steps performed in method 100, and may not recite the complete process or all steps of the method. Although various steps of method 100 are described below and depicted in FIG. 1, the steps need not necessarily all be performed, and in some cases may be performed simultaneously or in a different order than the order shown. In FIG. 1, some steps are illustrated in dashed lines indicating that such steps may be optional or may correspond to a specific example of a method according to the present disclosure.

In some examples, method 100 includes step 102, which includes receiving or obtaining a three-dimensional scan of a selected anatomical region for which the wearable device is configured to be utilized. For example, in the case in which the wearable device comprises an ankle-foot orthosis, step 102 may comprise obtaining a scan of the lower portion of a leg.

The scan may be obtained through the use of one or more imaging devices suitable for producing three-dimensional scans of the selected anatomical region. For example, the three-dimensional scan may be obtained through the use of an infrared (IR) light sensor, visible light sensor, etc. In some examples, the three-dimensional scan is obtained through the use of photogrammetry. Additionally, or alternatively, the scan may be obtained through the use of structured light scanning, laser scanning, and/or depth sensing camera(s). In some examples, step 102 may include receiving the three-dimensional scan, e.g., via network communication. In some examples, step 102 includes receiving a pre-existing three-dimensional scan of an anatomical region, such as one previously obtained by an above-described process.

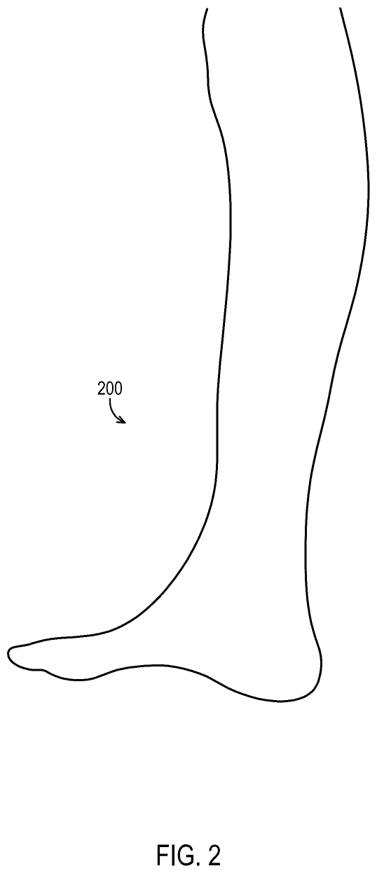

Step 104 of method 100 includes converting the three-dimensional scan into a three-dimensional surface model. An example three-dimensional surface model 200 is shown in FIG. 2. In some examples, step 104 includes several sub-steps, which may be accomplished automatically.

For example, step 104 may include generating a point cloud from the three-dimensional scan, the point cloud comprising a set of points in three-dimensional space corresponding to respective positions on the surface of the scanned anatomical region. In such examples, surface reconstruction may be accomplished through the use of mesh generation based on the point cloud, such as through the use of Delaunay Triangulation, Poisson Surface Reconstruction, or other suitable mesh generation technique(s).

Step 104 may include refining and/or smoothing the surface model. For example, if the scan obtained in step 102 has missing data, e.g., due to occlusions and/or limitations of the scanning device, one or more surface smoothing techniques, such as Laplacian smoothing may be utilized. In some examples, one or more further refining and/or smoothing techniques may be used to further refine the surface model, such as denoising, filtering, and/or fairing. Additionally, or alternatively, further refinement of the surface model may include hole filling, topology correction, or other suitable geometry correcting techniques.

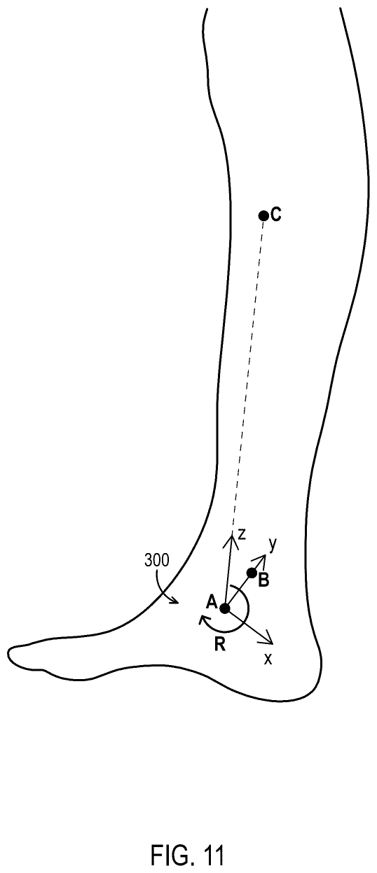

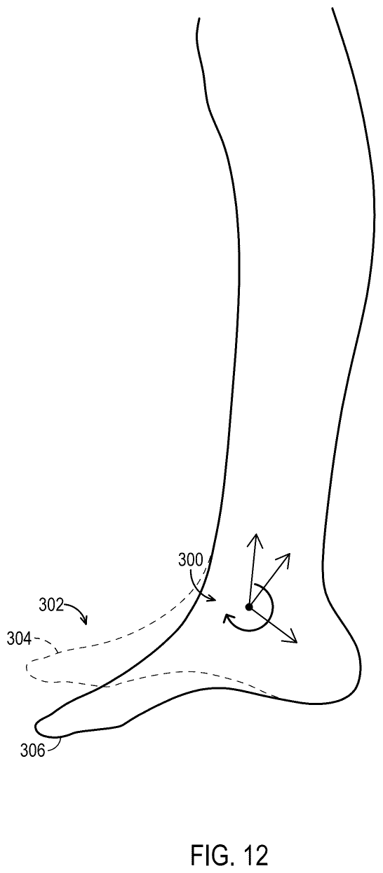

Step 105 may include virtually realigning the surface model, e.g., along defined joint axes. Examples of defined joint axes are shown in FIGS. 11 and 12. As shown in FIG. 11, step 105 may include defining one or more axes of rotation using the digital and/or anatomical landmarks. For example, general landmarks may be chosen for their relevance to larger joint motion, e.g., a knee, an elbow, an ankle, etc. In some examples, specific anatomical landmarks, such as a medial and/or lateral malleolus, can provide more precise references for defining the axes of rotation.

Step 105 may include virtually rotating a portion of the surface model relative to another portion around selected axes. Digital distortion reduction techniques can be applied to the surface model post-realignment. In other words, after aligning and rotating portions of the wearable device model, any distortions introduced during the process may be corrected, such as through the use of mesh smoothing, etc.

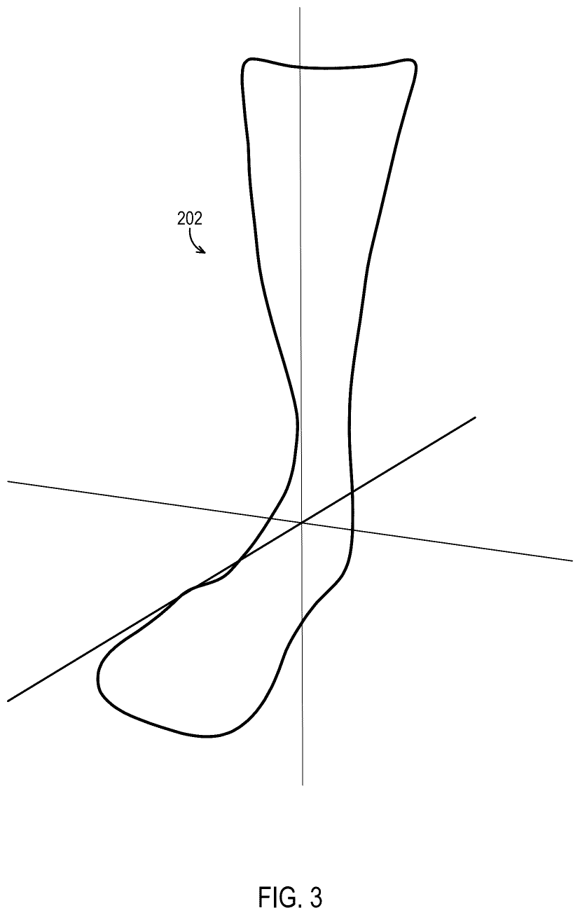

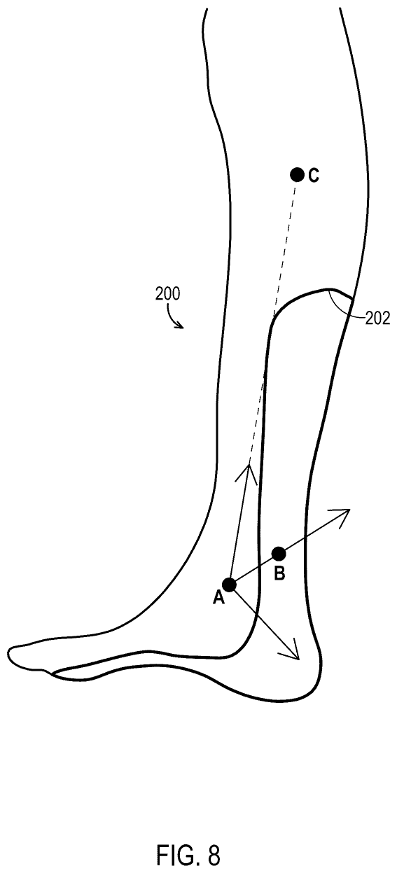

Step 106 of method 100 includes mapping a three-dimensional curve corresponding to the desired wearable device onto the surface of the surface model. An example of a three-dimensional curve 202 corresponding to an ankle-foot orthosis is shown in FIGS. 3-7. In general, the three-dimensional curve comprises a closed curve having a contour which follows a general edge boundary of the desired wearable device. The mapping process utilized in step 106 may include aligning and/or scaling the three-dimensional curve to the surface model using one or more digital landmarks. An example of digital landmarks utilized in step 106 is shown at A, B, and C in FIG. 8.

In some examples, the one or more digital landmarks may be manually defined, e.g., by a user. For example, the user may select one or more positions on the surface model to define the digital landmarks such that step 106 includes mapping the curve to the surface model based, at least in part, on the manually selected landmarks. As an example, the user may select a region of the surface model corresponding to a desired top edge of the resulting wearable device. Accordingly, the curve may be aligned and/or scaled to the surface model such that the portion of the curve corresponding to the top edge of the wearable device is located at the selected region. In some examples, the digital landmarks are automatically defined, e.g., based on one or more design criteria. For example, the digital landmarks may be automatically defined using one or more computer vision and/or image recognition techniques. Additionally, or alternatively, one or more digital landmarks may be automatically defined based on one more manually defined digital landmarks. For example, a user may manually select one or more digital landmarks, and in response, one or more further digital landmarks may be automatically defined.

Additionally, or alternatively, the mapping process utilized in step 106 includes aligning and/or scaling the three-dimensional curve to the surface model using one or more anatomical landmarks. In some examples, the anatomical landmarks may be manually selected in a manner substantially similar to the above-described digital landmarks.

In some examples, the one or more anatomical landmarks may be automatically determined based on the desired wearable device, e.g., through the use of one or more computer vision and/or image recognition techniques. Continuing the example of an ankle-foot orthosis, one or more computer vision and/or image recognition techniques may be used to automatically identify anatomical landmarks such as the medial malleolus, lateral malleolus, the heel, one or more toes, etc. These anatomical landmarks can serve as reference points for accurately positioning the three-dimensional curve onto the surface model such that the shape of the resulting wearable device accurately conforms to the anatomical structure of the wearer.

After the digital and/or anatomical landmarks are defined, the three-dimensional curve can be aligned to conform to the landmarks. For example, the three-dimensional curve may be rotated and/or linearly translated to conform to the landmarks.

Additionally, or alternatively, step 106 may include scaling the three-dimensional curve to match the proportions of the surface model while maintaining the general design and shape of the wearable device. Scaling the three-dimensional curve may include adjusting the size and/or shape of one or more portions of the curve to fit within the boundaries of the scanned region and/or conform to the one or more digital and/or anatomical landmarks.

In some examples, mapping the three-dimensional curve to the surface model includes mapping individual points along the curve to corresponding points on the surface model, such as through the use of projection, closest point mapping, ray casting, etc. For example, after aligning and/or scaling the curve to conform to the landmarks, the three-dimensional curve may be parameterized such that a series of points along the line may be iterated through, with each point along the line being mapped to the closest point on the surface model, e.g., in a direction normal to the surface model. Additionally, or alternatively, the curve may be uniformly projected, e.g., without parameterized iteration, onto to the surface of the model. An example of curve 202 after being mapped to the surface of surface model 200 is shown in FIG. 8.

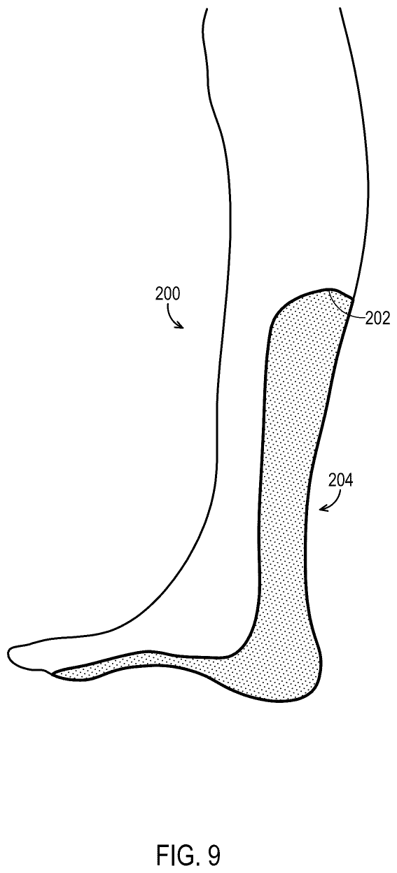

Step 108 of method 100 includes utilizing the mapped curve on the surface model to define the edge boundaries of a wearable device model. Stated differently, the shape of a virtual model of the wearable device is defined by the region of the surface model bound by the mapped curve. An example wearable device model having edge boundaries defined by a mapped curve is shown in the highlighted region 204 of FIG. 9.

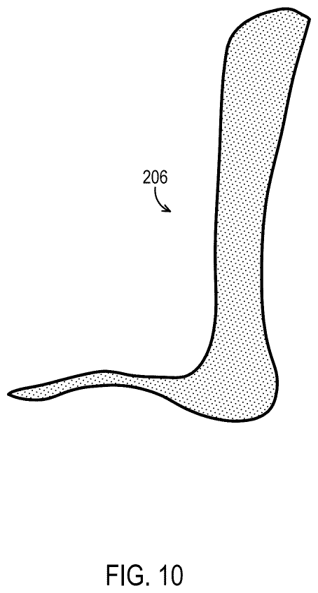

Step 110 of method 100 includes trimming away, i.e., removing, excess portions of the surface model outside of the defined edge boundaries, thereby defining the wearable device model. Accordingly, the resulting wearable device model is custom-fit to the surface model and, therefore, the anatomical region of which the original scan was taken. An example of a virtual model of a wearable device 206 is shown in FIG. 10.

In some examples, method 100 includes step 112, which includes adding thickness to the wearable device model such that the wearable device model is suitable for additive manufacturing. In some examples, the specific amount of thickness added to the wearable device model depends on the material to be used in manufacturing as well as the intended use of the device. For example, a wearable device configured to restrict motion may be less thick than a wearable device configured to provide protection from impact/abrasion. In general, the added thickness is added to the outward facing surface of the wearable device model, such that the inward facing (i.e., body facing) surface of the wearable device model is unchanged. In other words, the added thickness may be added to the surface of the wearable device model that will face away from the wearer, while the surface of the wearable device model that will face and/or contact the wearer remains unmodified.

In some examples, method 100 includes adding an offset to the wearable device model in a direction normal to the surface at step 114. In some examples, the offset is applied uniformly to the entirety of the wearable device model. In some examples, the offset is applied to selected portions of the wearable device model. In general, the offset is added in an outward facing direction (i.e., away from the wearer), such that the wearable device model retains the same shape and contour but is slightly larger by the amount of the offset.

In some examples, offset portions of the wearable device model may be configured to receive one or more additional materials, e.g., after additive manufacturing. For example, a wearable device may have an added offset portion suitable for receiving foam, padding, etc., after a shell of the wearable device is manufactured. In another example, the offset portion may be infilled with a different material and/or infill pattern during additive manufacturing.

Method 100 may include adding one or more additional features and/or functionalities to the wearable device model. For example, one or more strap slots, or other suitable fastener mounts may be added to the wearable device model. In general, the additional features and/or functionalities depend on the intended use and design of the wearable device. Method 100 may also include adding flared edges and/or raised internal surfaces to improve comfort and ease of use.

In some examples, method 100 includes step 116, which includes adding a range of motion to the wearable device, e.g., along defined joint axes, such as those shown at 300 in FIGS. 11 and 12. In some examples, step 116 includes incorporating a range of motion into the wearable device along and/or about defined joint axes through the use of flexible materials, mechanical structures, and/or compliant mechanisms.

As shown in FIG. 12, anatomically feasible movement of a respective body region can be simulated with the wearable device model, thereby modeling the natural flexion, extension, and rotation movements of the body region. In some examples, a specific amount of desired rotation about the joint axes can be selected by a user and/or automatically assigned based on the intended use of the wearable device. For example, a user might adjust the rotation limits for an ankle-foot orthosis to ensure it allows sufficient plantarflexion while restricting excessive inversion.

Step 116 may include virtually rotating a portion of the wearable device model relative to another portion around selected axes, thereby simulating the motion of the wearable device in relation to the body. In the example depicted in FIG. 12, a body region 302 is shown rotated about one or more axes of joint axes 300, from a first position 304 to a second position 306.

Digital distortion reduction techniques can be applied to the wearable device model post-realignment. In other words, after aligning and rotating portions of the wearable device model, any distortions introduced during the process may be corrected, such as through the use of mesh smoothing, etc.

Flexibility along the desired axes may be incorporated into the wearable device model, e.g., by combining rigid and flexible components, and/or incorporating compliant mechanisms/geometry. In some examples, this may include identifying portions of the wearable device model in which alterations to the material used in additive manufacturing, shell thickness, geometry, infill density, and/or infill pattern may be applied. For example, using a flexible polymer with a varying thickness may be used to provide more bending in certain areas while maintaining rigidity in others. In some examples, flexible regions of the wearable device model may be patterned with specific infill designs to allow for controlled stretching and/or compression. Additionally, or alternatively, one or more rigid components may incorporate structural features that limit specific ranges of motion or degrees of freedom. Furthermore, a transition region may be defined between rigid and flexible/compliant components, e.g., comprising a combination of both structural types, to facilitate smooth mechanical integration.

In some examples, method 100 includes step 118, which includes manufacturing the wearable device via additive manufacturing, e.g., fused deposition modeling, stereolithography, laser sintering, etc., based on the wearable device model. In some examples, certain portions of the wearable device, such as rigid portions, may be manufactured with certain method(s) of additive manufacturing and/or material(s), while other portions, such as flexible portions, may be manufactured with other method(s) of additive manufacturing and/or material(s). In general, step 118 comprises any suitable additive manufacturing process which results in a wearable device having the desired features enabled by the above-described methods.

Aspects of the design and manufacture of custom-fit wearable protective devices may be embodied as a computer method, computer system, or computer program product. Accordingly, aspects of the disclosed methods may take the form of an entirely hardware embodiment, an entirely software embodiment (including firmware, resident software, micro-code, and the like), or an embodiment combining software and hardware aspects, all of which may generally be referred to herein as a “circuit,” “module,” or “system.” Furthermore, aspects of the disclosed methods may take the form of a computer program product embodied in a computer-readable medium (or media) having computer-readable program code/instructions embodied thereon.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable non-transitory, tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of the design and manufacturing of wearable devices may be written in one or any combination of programming languages, including an object-oriented programming language (such as Java, C++), conventional procedural programming languages (such as C), and functional programming languages (such as Haskell). Mobile apps may be developed using any suitable language, including those previously mentioned, as well as Objective-C, Swift, C#, HTML5, and the like. The program code may execute entirely on a users computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), and/or the connection may be made to an external computer (for example, through the Internet using an Internet Service Provider).

Aspects of the design and manufacturing of custom-fit wearable devices may be described with reference to flowchart illustrations and/or block diagrams of methods, apparatuses, systems, and/or computer program products. Each block and/or combination of blocks in a flowchart and/or block diagram may be implemented by computer program instructions. The computer program instructions may be programmed into or otherwise provided to processing logic (e.g., a processor of a general purpose computer, special purpose computer, field programmable gate array (FPGA), or other programmable data processing apparatus) to produce a machine, such that the (e.g., machine-readable) instructions, which execute via the processing logic, create means for implementing the functions/acts specified in the flowchart and/or block diagram block(s).

Additionally or alternatively, these computer program instructions may be stored in a computer-readable medium that can direct processing logic and/or any other suitable device to function in a particular manner, such that the instructions stored in the computer-readable medium produce an article of manufacture including instructions which implement the function/act specified in the flowchart and/or block diagram block(s).

The computer program instructions can also be loaded onto processing logic and/or any other suitable device to cause a series of operational steps to be performed on the device to produce a computer-implemented process such that the executed instructions provide processes for implementing the functions/acts specified in the flowchart and/or block diagram block(s).

Any flowchart and/or block diagram in the drawings is intended to illustrate the architecture, functionality, and/or operation of possible implementations of systems, methods, and computer program products according to aspects of the present disclosure. In this regard, each block may represent a module, segment, or portion of code, which comprises one or more executable instructions for implementing the specified logical function(s). In some implementations, the functions noted in the block may occur out of the order noted in the drawings. For example, two blocks shown in succession may, in fact, be executed substantially concurrently, or the blocks may sometimes be executed in the reverse order, depending upon the functionality involved. Each block and/or combination of blocks may be implemented by special purpose hardware-based systems (or combinations of special purpose hardware and computer instructions) that perform the specified functions or acts.

### B. Selected Embodiments and Claim Concepts

This section describes additional aspects and features of the design and manufacture of custom-fit wearables, presented without limitation as a series of paragraphs, some or all of which may be alphanumerically designated for clarity and efficiency. Each of these paragraphs can be combined with one or more other paragraphs, and/or with disclosure from elsewhere in this application in any suitable manner. Some of the paragraphs below may expressly refer to and further limit other paragraphs, providing without limitation examples of some of the suitable combinations.
A0. A product comprising any feature described herein, either individually or in combination with any other such feature, in any configuration.
B0. A process for designing and manufacturing a custom-fit wearable device, the process comprising any process step described herein, in any order, using any modality.
C0. A method of manufacturing a custom-fit wearable device, the method comprising:
   identifying one or more anatomical landmarks on a three-dimensional model of a body portion;
   mapping a curve onto the three-dimensional model based at least in part on the one or more anatomical landmarks, such that a surface of a wearable device model is defined by a continuous area of the three-dimensional model bound by the curve;
   adding thickness to the surface of the wearable device model; and
   controlling an additive manufacturing process to manufacture the custom-fit wearable device based on the wearable device model.
C1. The method of paragraph C0, wherein the curve corresponds to a selected orthosis design.
C2. The method of paragraph C0 and/or C1, wherein the mapping the curve onto the three-dimensional model comprises projecting portions of the curve onto the three-dimensional model.
C3. The method of any of paragraphs C0-C2, wherein the curve is a closed curve.
C4. The method of paragraph C3, further comprising adding an offset to the wearable device model.
C5. The method of paragraph C4, wherein the controlling the additive manufacturing process comprises controlling the additive manufacturing process to manufacture a liner within the offset.
C6. The method of any of paragraphs C0-C5, wherein a selected portion of the custom-fit wearable device has increased flexibility.
C7. The method of paragraph C6, wherein the selected portion comprises a flexible material.
C8. The method of paragraphs C6 and/or C7, wherein the selected portion comprises a compliant mechanism.
C9. The method of any of paragraphs C0-C8, further comprising scanning an anatomical region to generate the three-dimensional model of the body portion.
D0. A method of manufacturing a custom-fit wearable device comprising:
identifying one or more anatomical landmarks on a three-dimensional model of a body portion;
mapping a closed curve onto the three-dimensional model based at least in part on the one or more anatomical landmarks, such that a surface of the custom-fit wearable device is defined by a continuous portion of the three-dimensional model bound by the closed curve; and
manufacturing the custom-fit wearable device via additive manufacturing.
D1. The method of D0, further comprising imagine an anatomical region to generate the three-dimensional model of the body portion.
E0. A method for creating a standard-design, custom-fit wearable device comprising:
obtaining a scan of an anatomical region;
converting the scan to a continuous surface model;
aligning and scaling a three-dimensional curve corresponding to the custom-fit wearable device to the continuous surface model based on digital and/or anatomical landmarks selected by a user;
defining edge boundaries of the custom-fit wearable device based on the three-dimensional curve;
trimming the continuous surface model to the edge boundaries to define a trimmed surface; and
applying thickness and offset to the trimmed surface to generate a virtual model of the custom-fit wearable device.
E1. The method of paragraph E0, wherein the custom-fit wearable device may receive features, such as strap slots, according to a standard design for a standard wearable device.
E2. The method of paragraph E0 and/or E1, wherein the aligning, scaling, and/or projecting of the three-dimensional curve to the continuous surface is accomplished automatically by a computer device.
E3. The method of any of paragraphs E0-E2, wherein the custom-fit wearable device is additively manufactured.
F0. A method for virtually realigning joint orientation of a surface model of a body region for a custom-fit wearable device, the method comprising:
locating joint axes of rotation by digital and/or anatomical landmarks on the surface model;
defining a rotation range of the joint axes of rotation by anatomically feasible movement of the body region;
receiving user input on the amount and direction of desired rotation about a specified joint axis;
virtually rotating a portion of the surface model about the specified joint axis; and
after the virtually rotating, utilizing one or more digital distortion techniques on the surface model.
F1. The method of paragraph F0 further comprising manufacturing the custom-fit wearable device by additively manufacturing the surface model.
G0. A method of manufacturing a custom-fit wearable device, the method comprising:
identifying one or more anatomical landmarks on a three-dimensional model of an anatomical body portion;
mapping a curve onto the three-dimensional model based at least in part on the one or more anatomical landmarks, such that a surface of a wearable device model is defined by a continuous area of the three-dimensional model bound by the curve;
adding thickness to the surface of the wearable device model; and
controlling an additive manufacturing process to manufacture the custom-fit wearable device based on the wearable device model.
G1. The method of paragraph G0, wherein the curve corresponds to a selected orthosis design.
G2. The method of paragraphs G0 and/or G1, wherein the mapping the curve onto the three-dimensional model comprises projecting portions of the curve onto the three-dimensional model.
G3. The method of any one of paragraphs G0-G2, further comprising scanning an anatomical region to generate the three-dimensional model of the anatomical body portion.
G4. The method of any one of paragraphs G0-G3, further comprising adding an offset to the wearable device model.
G5. The method of paragraph G4, wherein the controlling the additive manufacturing process includes controlling the additive manufacturing process to manufacture a liner within the offset.
G6. The method of any one of paragraphs G0-G5, wherein a selected portion of the custom-fit wearable device has increased flexibility.
G7. The method of paragraph G6, wherein the selected portion comprises a flexible material.
G8. The method of paragraph G6 and/or G7, wherein the selected portion comprises a compliant mechanism.
H0. A data processing system for generating a wearable device model, the system comprising:
one or more processors;
a memory; and
a plurality of instructions stored in the memory and executable by the one or more processors to:
receive as input a scan of an anatomical region to generate a three-dimensional model of a body portion;
identify one or more anatomical landmarks on the three-dimensional model;
map a curve onto the three-dimensional model based at least in part on the one or more anatomical landmarks, such that a surface of a wearable device model is defined by a continuous area of the three-dimensional model bound by the curve;
add a thickness to the surface of the wearable device model; and
control an additive manufacturing process to manufacture a custom-fit wearable device based on the wearable device model.
H1. The data processing system of paragraph H0, wherein the curve corresponds to a selected orthosis design.
H2. The data processing system of paragraph H0 and/or H1, wherein the plurality of instructions is further executable by the one or more processors to map the curve onto the three-dimensional model by projecting portions of the curve onto the three-dimensional model.
H3. The data processing system of paragraph H2, wherein the curve is a closed curve.
H4. The data processing system of any one of paragraphs H0-H3, wherein the plurality of instructions is further executable by the one or more processors to add an offset to the wearable device model.
H5. The data processing system of paragraph H4, wherein the plurality of instructions is further executable by the one or more processors to control the additive manufacturing process to manufacture a liner within the offset.
H6. The data processing system of any one of paragraphs H0-H5, wherein a selected portion of the custom-fit wearable device has increased flexibility.
H7. The data processing system of paragraph H6, wherein the selected portion comprises a flexible material.
H8. The data processing system of paragraphs H6 and/or H7, wherein the selected portion comprises a compliant mechanism.
J0. A computer-implemented method of generating a virtual model of a wearable device, the method comprising:
receiving a scan of an anatomical region;
converting the scan to a continuous surface model;
aligning and scaling a three-dimensional curve corresponding to a wearable device to the continuous surface based on one or more landmarks of the continuous surface model;
defining edge boundaries of the wearable device based on the three-dimensional curve;
trimming the continuous surface model to the edge boundaries to define a trimmed surface; and
applying thickness and offset to the trimmed surface to generate a virtual model of the wearable device.
J1. The method of paragraph J0, further comprising controlling an additive manufacturing process to manufacture the wearable device based on the virtual model.

### Advantages, Features, and Benefits

The different embodiments and examples of the custom wearable device manufacturing methods described herein provide several advantages over known solutions for manufacturing custom devices. For example, illustrative embodiments and examples described herein allow precise and personalized fitting of devices based on detailed anatomical scans thereby ensuring optimal fit and effectiveness of the devices.

Additionally, and among other benefits, illustrative embodiments and examples described herein allow a more efficient and customizable manufacturing process using additive manufacturing.

Additionally, and among other benefits, illustrative embodiments and examples described herein utilize different materials and/or geometries for added functionality, such as increased and/or decreased flexibility/motion in specific areas.

Additionally, and among other benefits, illustrative embodiments and examples described herein provide a less computationally intensive (i.e., more efficient) method for manufacturing a custom-fit wearable device.

No known system or device can perform these functions. However, not all embodiments and examples described herein provide the same advantages or the same degree of advantage.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A computer implemented method of manufacturing a custom-fit wearable device, the method comprising:
identifying one or more anatomical landmarks on a three-dimensional model of an anatomical body portion;
mapping a curve onto the three-dimensional model based at least in part on the one or more anatomical landmarks, such that a surface of a wearable device model is defined by a continuous area of the three-dimensional model bound by the curve;
adding thickness to the surface of the wearable device model; and
controlling an additive manufacturing process to manufacture the custom-fit wearable device based on the wearable device model.

2. The computer implemented method of claim 1, wherein the curve corresponds to a selected orthosis design.

3. The computer implemented method of claim 1, wherein the mapping the curve onto the three-dimensional model comprises projecting portions of the curve onto the three-dimensional model.

4. The computer implemented method of claim 1, further comprising scanning an anatomical region to generate the three-dimensional model of the anatomical body portion.

5. The computer implemented method of claim 1, further comprising adding an offset to the wearable device model.

6. The computer implemented method of claim 5, wherein the controlling the additive manufacturing process includes controlling the additive manufacturing process to manufacture a liner within the offset.

7. The computer implemented method of claim 1, wherein a selected portion of the custom-fit wearable device has increased flexibility.

8. The computer implemented method of claim 7, wherein the selected portion comprises a flexible material.

9. The computer implemented method of claim 7, wherein the selected portion comprises a compliant mechanism.

10. A data processing system for generating a wearable device model, the system comprising:
one or more processors;
a memory; and
a plurality of instructions stored in the memory and executable by the one or more processors to:
receive as input a scan of an anatomical region to generate a three-dimensional model of a body portion;
identify one or more anatomical landmarks on the three-dimensional model;
map a curve onto the three-dimensional model based at least in part on the one or more anatomical landmarks, such that a surface of a wearable device model is defined by a continuous area of the three-dimensional model bound by the curve;
add a thickness to the surface of the wearable device model; and
control an additive manufacturing process to manufacture a custom-fit wearable device based on the wearable device model.

11. The data processing system of claim 10, wherein the curve corresponds to a selected orthosis design.

12. The data processing system of claim 10, wherein the plurality of instructions is further executable by the one of more processors to map the curve onto the three-dimensional model by projecting portions of the curve onto the three-dimensional model.

13. The data processing system of claim 12, wherein the curve is a closed curve.

14. The data processing system of claim 10, wherein the plurality of instructions is further executable by the one of more processors to add an offset to the wearable device model.

15. The data processing system of claim 14, wherein the plurality of instructions is further executable by the one of more processors to control the additive manufacturing process to manufacture a liner within the offset.

16. The data processing system of claim 10, wherein a selected portion of the custom-fit wearable device has increased flexibility.

17. The data processing system of claim 16, wherein the selected portion comprises a flexible material.

18. The data processing system of claim 16, wherein the selected portion comprises a compliant mechanism.

19. A computer implemented method of generating a virtual model of a wearable device, the method comprising:
receiving a scan of an anatomical region;
converting the scan to a continuous surface model;
aligning and scaling a three-dimensional curve corresponding to a wearable device to the continuous surface based on one or more landmarks of the continuous surface model;
defining edge boundaries of the wearable device based on the three-dimensional curve;
trimming the continuous surface model to the edge boundaries to define a trimmed surface; and
applying thickness and offset to the trimmed surface to generate a virtual model of the wearable device.

20. The computer implemented method of claim 19, further comprising controlling an additive manufacturing process to manufacture the wearable device based on the virtual model.
